(19) United States
(12) Patent Application Publication
(10) Pub. No.: US 2024/0092753 A1
(43) Pub. Date: Mar. 21, 2024

(54) PROCESS FOR PRODUCTION OF INTERMEDIATES

(71) Applicant: DSM IP Assets B.V., Heerlen (NL)

(72) Inventors: Werner BONRATH, Kaiseraugst (CH); Jonathan Alan MEDLOCK, Kaiseraugst (CH); Marc-André MUELLER, Kaiseraugst (CH); Bettina WUESTENBERG, Kaiseraugst (CH)

(21) Appl. No.: 18/261,182

(22) PCT Filed: Jan. 14, 2022
(86) PCT No.: PCT/EP2022/050708

§ 371 Date: Jul. 12, 2023

(30) Foreign Application Priority Data
Feb. 1, 2021 (EP) 21154605.6

(51) Int. Cl. C07D 333/48

(52) U.S. Cl. CPC C07D 333/48

(57) ABSTRACT

The present invention relates to a new process for the production of specific intermediates, which are preferably used in the production of vitamin A, vitamin A acetate, or β-carotene and derivatives thereof, e.g. canthaxanthin, astaxanthin or zeaxanthin.

The present invention relates to a new process for the production of specific intermediates (building blocks), which are preferably used in the production of vitamin A, vitamin A acetate, or β-carotene and derivatives thereof, e.g. canthaxanthin, astaxanthin or zeaxanthin.

Vitamin A or its derivatives such as Vitamin acetate

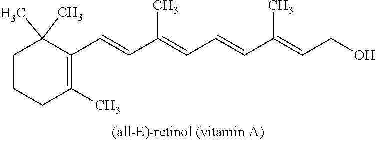 [SMILES: CC1=C(/C=C/C(C)=C/C=C/C(C)=C/CO)C(C)(C)CCC1]

is an important ingredient for many applications. Vitamin A plays a role in a variety of functions throughout the body, such as e.g. vision processes, gene transcription, immune function, bone metabolism, haematopoiesis, skin and cellular health and antioxidant functions.

Due to the importance of vitamin A (and its derivatives) and the complexity of the synthesis thereof, there is always a need for improved processes of production.

The goal of the present invention was to find easily accessible compounds, which can then be used in an improved synthesis of vitamin A or its derivates, or β-carotene, preferably vitamin A (acetate). The aim was achieved by the synthesis as disclosed and described below.

The intermediates, which are produced by the process according to the present invention are the compounds of formula (I)

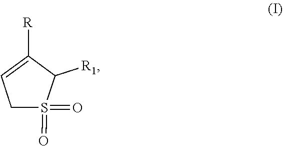

wherein
R is C₁C₄-alkyl, C₃-C₂₀-alkylene or a moiety of formula

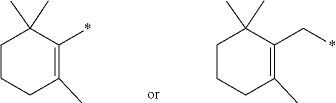

(the * shows where the moiety is attached),
R₁ is H, CH₂OR₂, wherein
R₂ is H or —(CO)R₃, wherein R₃ is a C₁-C₁₈-alkyl, or R₁ is a moiety of formula

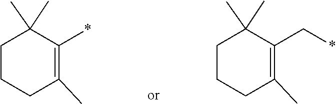

(the asterisk shows where the moiety is attached).

To obtain a compound of formula (I), the compound of formula (II)

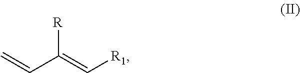

wherein R and R₁ have the same meanings as defined for the compound of formula (I)
is reacted with SO₂ in at least one alcohol as solvent.

The process according to the present invention is carried out without (in the absence) of any aromatic solvent and/or chlorinated solvent. This is a great advantage due to the ecological downside of such solvents.

Therefore, the present invention relates to a process (P) for the production of a compound of formula (I)

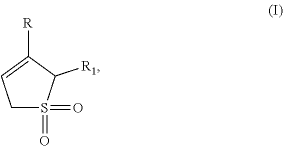

wherein
R is C₁-C₄-alkyl, C₃-C₂₀-alkylene or a moiety of formula

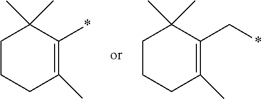

(the * shows where the moiety is attached),
R₁ is H, CH₂OR₂, wherein
R₂ is H or —(CO)R₃, wherein R₃ is a C₁-C₁₈-alkyl, or R₁ is a moiety of formula

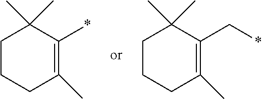

(the asterisk shows where the moiety is attached), characterized in that a compound of formula (II)

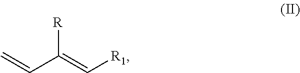

wherein R and R₁ have the same meanings as defined above is reacted with SO₂ in at least one alcohol as solvent.

As stated above, the reaction according to the present invention is carried out without (in the absence) of any aromatic solvent and/or chlorinated solvent. Such a solvent is used in an excess in regard to the reactants.

Therefore, the present invention relates to the process (P′), which is the process (P), wherein the process is carried out in the absence of any aromatic solvent and/or chlorinated solvent.

It is known from the prior art how to obtain the compounds of formula (II) (e. g. from Z. Wu et al, J. Am. Chem. Soc., 2005, 17433).

The following scheme illustrates how to obtain Vitamin A acetate starting from the compound of formula (I):

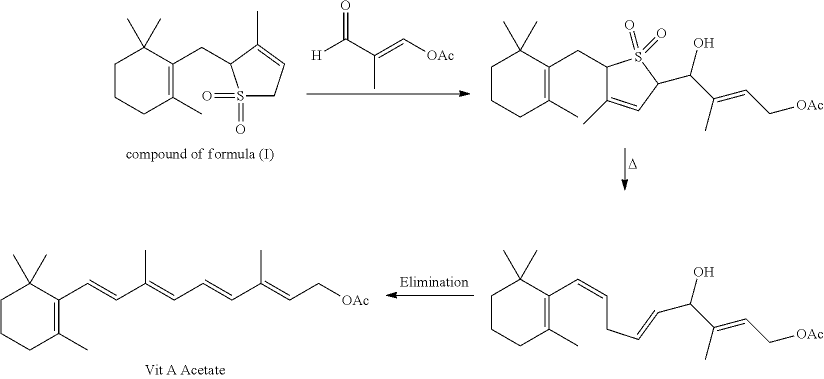

In a preferred embodiment, compounds of formula (I)

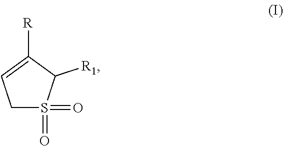

wherein
R is C₁-C₂-alkyl, C₃-C₁₆-alkylene or

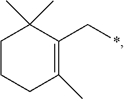

(the * shows where the moiety is attached),
R₁ is H, CH₂OR₂, wherein
R₂ is H or —(CO)R₃, wherein R₃ is a C₁-C₁₆-alkyl, or
R₁ is a moiety of formula

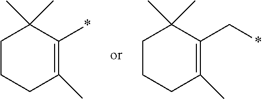

(the * shows where the moiety is attached),
are produced.

In a more preferred embodiment compounds of formula (I′)

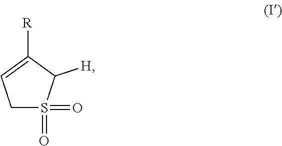

wherein R is CH₃, C₁₀-C₁₆-alkylene or

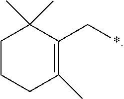

(the * shows where the moiety is attached),
are produced.

In another more preferred embodiment compounds of formula (I″)

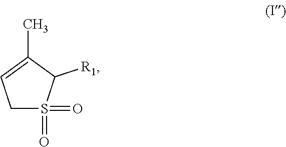

wherein R₁ is CH₂OR₂, wherein
R₂ is H or —(CO)R₃, wherein R₃ is a C₁-C₁₆-alkyl, or
R₁ is a moiety of formula

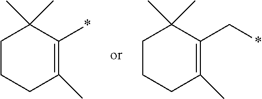

(the * shows where the moiety is attached),
are produced.

The most preferred compounds which are produced by the process according to the present invention are the following

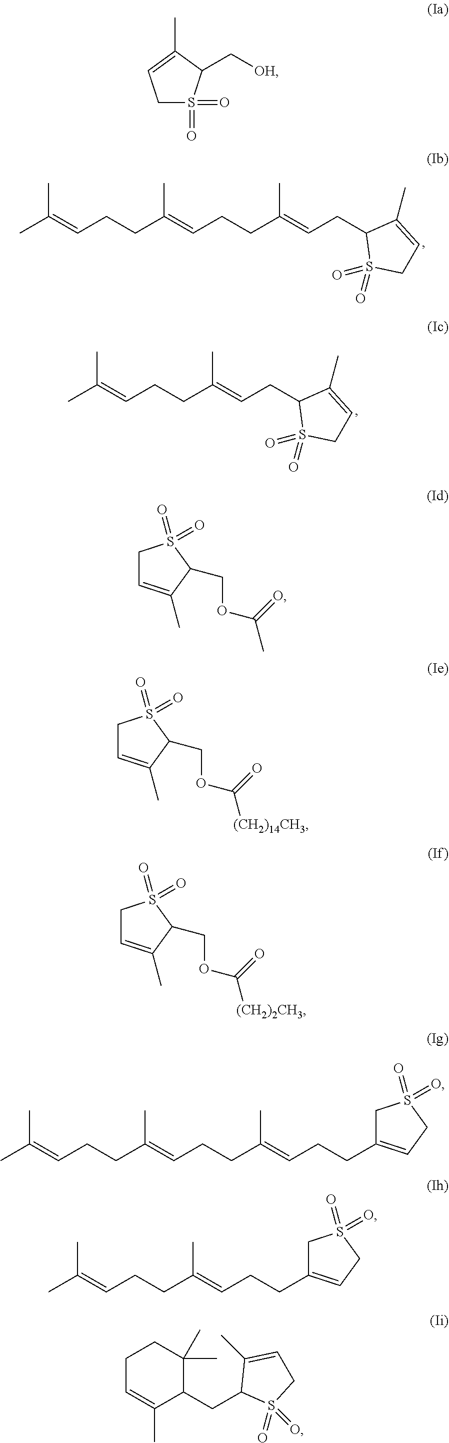

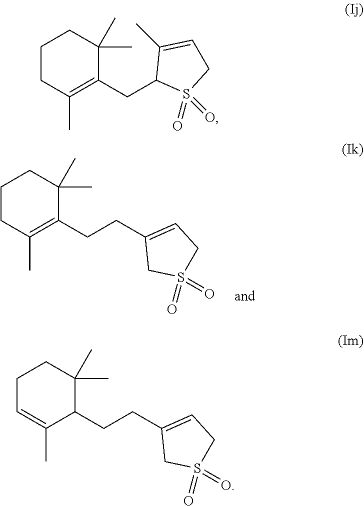

Therefore, the present invention relates to a process (P″), which is process (P) or (P′), wherein
R is C₁-C₂-alkyl, C₃-C₁₆-alkylene or

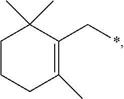

(the * shows where the moiety is attached),
R₁ is H, CH₂OR₂, wherein
R₂ is H or —(CO)R₃, wherein R₃ is a C₁-C₁₆-alkyl, or
R₁ is a moiety of formula

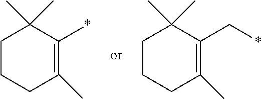

(the * shows where the moiety is attached).

Therefore, the present invention relates to a process (P′″), which is process (P) or (P′), wherein compounds of formula (I′)

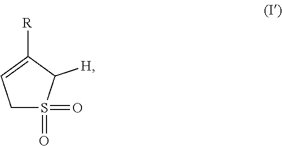

wherein R is CH₃, C₁₀-C₁₆-alkylene or

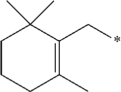 [SMILES: *CC1=C(C)CCCC1(C)C]

(the * shows where the moiety is attached),
are produced.

Therefore, the present invention relates to a process (P″″), which is process (P) or (P′), wherein compounds of formula (I″)

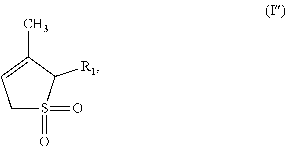

wherein R₁ is CH₂OR₂, wherein
R₂ is H or —(CO)R₃, wherein R₃ is a C₁-C₁₆-alkyl, or
R₁ is a moiety of formula

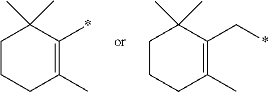

(the * shows where the moiety is attached),
are produced.

Therefore, the present invention relates to a process (P′″″), which is process (P) or (P′), wherein the following compounds are produced

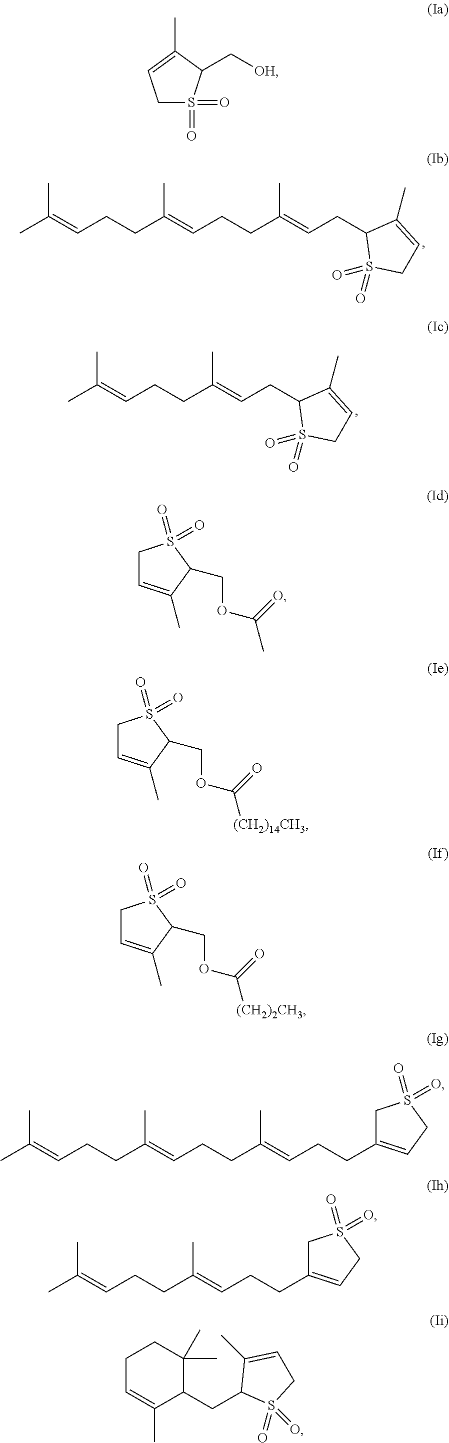

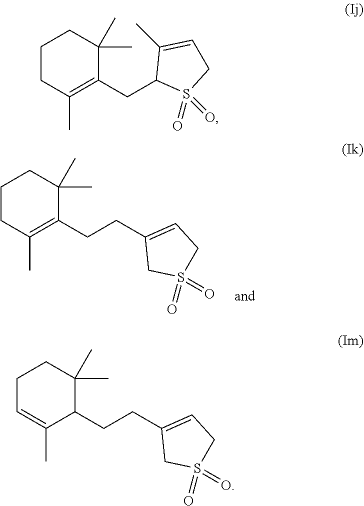

The process according to the present invention is carried out in an inert solvent, which is an alcohol. Preferably the alcohol is chosen from the group consisting of methanol, ethanol, 1-propanol, 2-propanol, tert-butanol, and ethylene glycol.

Therefore, the present invention relates to the process (P1), which is process (P), (P′), (P″), (P′″), (P″″) or (P′″″), wherein the alcohol is chosen from the group consisting of methanol, ethanol, 1-propanol, 2-propanol, tert-butanol and ethylene glycol.

The process according to the present invention is usually carried out at elevated temperatures. Usually, a temperature range of from 20° C. to 125° C., preferably at a temperature range of from 25° C. to 110° C.

Therefore, the present invention relates to the process (P2), which is process (P), (P′), (P″), (P′″), (P″″), (P′″″) or (P1), wherein the process is carried out at a temperature range of from 20° C. to 125° C.

Therefore, the present invention relates to the process (P2′), which is process (P), (P′), (P″), (P′″), (P″″), (P′″″) or (P1), wherein the process is carried out at a temperature range of from 25° C. to 110° C.

SO₂ can be added to the reaction mixture in gaseous form. It can be pure SO₂ gas as well as a suitable mixture comprising SO₂ gas. It is also possible to add the SO₂ to the reaction mixture as a liquid, either at reduced temperature or at elevated pressures. The desired reaction is independent of how the SO₂ is introduced into the reaction mixture.

The reaction can be carried out at ambient pressure or at elevated pressure.

It is possible to carry out the invention adding the SO₂ gas into the reaction mixture continuously or by adding the desired amount of SO₂ to reaction mixture at once (or in several portions) at any time.

Preferably the SO₂ is given to reaction mixture at the start and the reaction is carried out at an elevated pressure.

When elevated pressure is used, the pressure is 0.5-5 barg, preferably 0.5-3 barg. barg is called the gauge pressure and it is defined a defined as the difference between the absolute pressure (P_{abs}) and the prevailing atmospheric pressure (P_{amb}).

Therefore, the present invention relates to the process (P3), which is process (P), (P′), (P″), (P′″), (P″″), (P′″″), (P1), (P2) or (P2′), wherein the process is carried out at an elevated pressure.

Therefore, the present invention relates to the process (P3′), which is process (P3), wherein the gauge pressure is between 0.5 to 5 barg.

Therefore, the present invention relates to the process (P3″), which is process (P3), wherein the gauge pressure is between 0.5 to 3 barg.

The starting material, which are the compounds of formula (I) and the SO₂ can be used in equimolar amounts. But it is also possible to use an excess of one of the starting material. Usually, SO₂ is added in excess.

Therefore, the present invention relates to the process (P4), which is process (P), (P′), (P″), (P′″), (P″″), (P′″″), (P1), (P2), (P2′), (P3), (P3′) or (P3″), wherein the compounds of formula (I) and SO₂ are used in equimolar amount.

Therefore, the present invention relates to the process (P4′), which is process (P), (P′), (P″), (P′″), (P″″), (P′″″), (P1), (P2), (P2′), (P3), (P3′) or (P3″), wherein SO₂ is used in excess in view of the compounds of formula (I).

Furthermore, the process according to the present invention can also be carried out in the presence of at least one stabilizer. Such stabilizers are well known. Suitable stabilizers are i.e. butylated hydroxytoluene (BHT), phenothiazine, 4-tert-butylcatechol and tocopherol. These compounds are added in small amounts.

Therefore, the present invention relates to the process (P5), which is process (P), (P′), (P″), (P′″), (P″″), (P′″″), (P1), (P2), (P2′), (P3), (P3′), (P3″), (P4) or (P4′), wherein the process is carried out in the presence of at least one stabilizer.

Therefore, the present invention relates to the process (P5′), which is process (P5), wherein the at least one stabilizer is chosen from the group consisting of butylated hydroxytoluene (BHT), phenothiazine, 4-tert-butylcatechol and tocopherol.

The obtained products of the process according to the present invention (these are the compound of formula (I)) are ideal intermediates. Especially in the production of vitamin A and its derivates. Some of the obtained compounds of formula (I) are new.

Therefore, the present invention relates to the following compounds

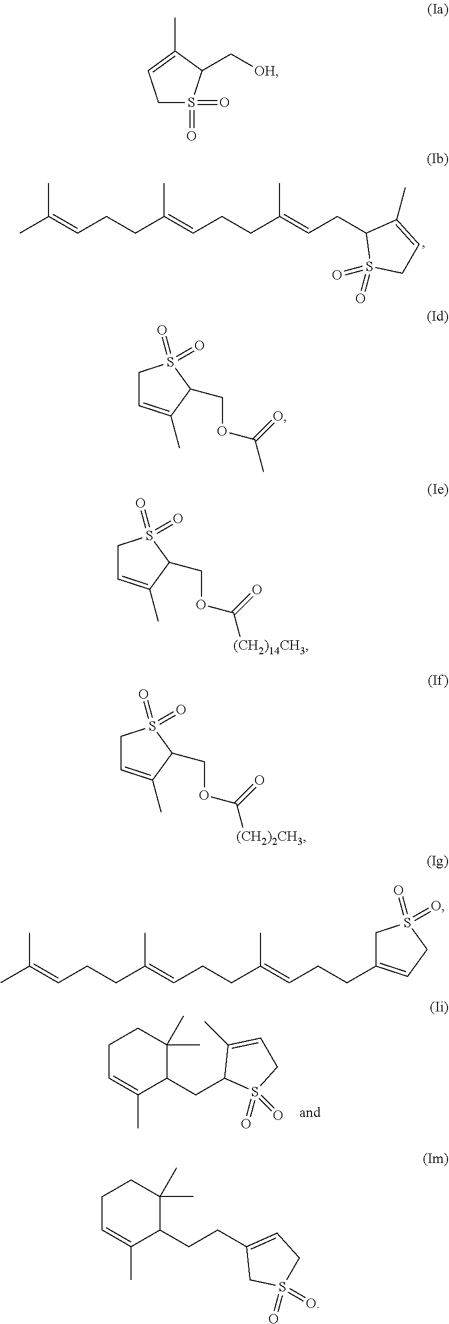

Alternatively, the compound of formula (I′″)

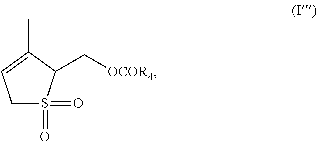

wherein R₄ is a C₁-C₁₈-alkyl,

can also be produced by using the compound of formula (Ia) as starting material and then conversion into the compound of formula (I′″) with a suitable reagent, such as (but not limited to) an acid chloride or an anhydride.

As stated above the compounds of formula (I) are suitable intermediates in organic synthesis, especially for the synthesis of vitamin A, vitamin A acetate, or β-carotene and derivatives thereof, e.g. canthaxanthin, astaxanthin or zeaxanthin.

The following example serve to illustrate the invention. The temperature is given in ° C. and all percentages are related to the weight.

### EXAMPLES

### Example 1

(E)-1,3,3-trimethyl-2-(3-methylpenta-2,4-dien-1-yl)cyclohex-1-ene (7.5 g, 0.03 mol), BHT (125 mg, 0.0005 mol) and methanol (40 ml, 1.0 mol) were placed in a 125 ml Hastelloy reactor. The reaction mixture purged with nitrogen and sulfur dioxide gas was introduced from a cylinder until the internal pressure was approximately 1.5 barg. The reaction mixture was heated at 70° C. for 14 hours and cooled to room temperature. The pressure was released, the reactor was purged with nitrogen and all volatile material was evaporated under reduced pressure to obtain the crude product as a dark solid (9.6 g).

The crude product was purified by crystallization from methanol/n-hexane, giving 7.1 g of (Ii) as a light grey solid (purity 95.6% [w %], yield 84%).

¹H NMR (300 MHz, CHLOROFORM-d) δ ppm 1.03 (s, 3H), 1.10 (s, 3H), 1.43-1.49 (m, 2H), 1.59 (s, 3H), 1.60-1.68 (m, 2H), 1.83-1.89 (m, 3H), 1.90-1.99 (m, 2H), 2.50-2.61 (m, 1H), 2.66-2.76 (m, 1H), 3.60-3.79 (m, 2H), 3.65-3.69 (m, 1H), 5.70 (dt, J=3.6, 1.6 Hz, 1H)

¹³C NMR (75 MHz, CHLOROFORM-d) δ ppm 19.2 (s), 20.1 (s), 20.9 (s), 28.1 (s), 28.8 (s), 29.8 (s), 33.0 (s), 35.0 (s), 40.0 (s), 54.2 (s), 65.9 (s), 117.8 (s), 131.8 (s), 131.8 (s), 141.7 (s)

### Example 2

(E)-3-Methylpenta-2,4-dien-1-ol (10 g, 0.1 mol), BHT (250 mg, 0.001 mol) and methanol (40 ml, 1.0 mol) were placed in a 125 ml Hastelloy reactor. The reaction mixture purged with nitrogen and sulfur dioxide gas was introduced from a cylinder until the internal pressure was approximately 1.5 barg. The reaction mixture was heated at 70° C. for 14 hours and cooled to room temperature. The pressure was released, the reactor was purged with nitrogen and all volatile material was evaporated under reduced pressure to obtain the product as a very dark oil (15.2 g).

The dark oil (15.2 g) was dissolved in dichloromethane (20 ml) and filtered through a silica gel pad (50 g, d=4.0 cm), washing with heptane/ethyl acetate. The product filtrate was evaporated at 40° C. (5 mbar) to obtain the crude product as a brown oil (12.9 g).

The crude product was purified by crystallization in toluene to yield 9.1 g of (1a) as a light grey solid, (56%).

1H NMR (300 MHz, CHLOROFORM-d) δ ppm 1.90 (m, J=2.1, 0.9 Hz, 3H), 2.86 (br. s., 1H), 3.63 (m, 1H), 3.76 (m, 2H), 3.95 (dd, J=12.7, 5.7 Hz, 1H), 4.17 (d, J=11.5 Hz, 1H), 5.79 (m, J=2.9, 1.4 Hz, 1H).

13C NMR (75 MHz, CHLOROFORM-d) δ ppm 17.9 (s), 56.4 (s), 58.5 (s), 69.2 (s), 118.9 (s), 135.6 (s).

### Example 3

(E)-3-Methylpenta-2,4-dien-1-yl acetate (10 g, 0.07 mol), BHT (250 mg, 0.001 mol) and methanol (40 ml, 1.0 mol) were placed in a 125 ml Hastelloy reactor. The reaction mixture purged with nitrogen and sulfur dioxide gas was introduced from a cylinder until the internal pressure was approximately 1.5 barg. The reaction mixture was heated at 70° C. for 14 hours and cooled to room temperature. The pressure was released, the reactor was purged with nitrogen and all volatile material was evaporated under reduced pressure to obtain the product (Ii) as a very dark oil (12.5 g).

The crude product was purified by a column chromatography to obtain the product as a slightly yellow oil (6.15 g, 43%).

¹H NMR (300 MHz, CHLOROFORM-d) δ ppm 1.88-1.92 (m, 3H), 2.09 (s, 3H), 3.66-3.82 (m, 3H), 4.44-4.57 (m, 2H), 5.81 (m, 1H).

¹³C NMR (75 MHz, CHLOROFORM-d) δ ppm 18.1 (s), 20.7 (s), 56.2 (s), 59.6 (s), 66.09 (s), 119.5 (s), 135.3 (s), 170.3 (s).

### Example 4

2-(Hydroxymethyl)-3-methyl-2,5-dihydrothiophene 1,1-dioxide (Ia) (3.96 g, 24.4 mmol, 1.0 eq), acetic anhydride (3.50 mL, 36.6 mmol, 1.5 eq, d=1.081 g/mL) and pyridine (39 μl, 0.49 mmol, 0.02 eq, d=0.978 g/mL) were placed in a dried three necked round bottom flask with a magnetic stirrer under an argon atmosphere. The reaction mixture was stirred at room temperature for 2 h.

Water (50 mL) and ethyl acetate (50 mL) were added to the reaction mixture and stirred for 30 min. The aqueous layer was separated and extracted with ethyl acetate (2×50 mL). The organic layers were washed with water (30 mL) and with a saturated sodium hydrogen carbonate solution (35 mL). Afterwards, combined organic layers were filtered over cotton wool and evaporated at 45° C. (2 mbar) to obtain (Ii) as a yellow oil, which crystallized slowly (4.19 g). Yield: 84%.

1. Process of the production of compounds of formula (I) 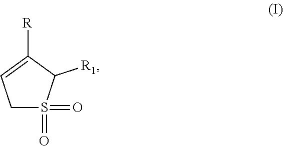
wherein
R is C₁-C₄-alkyl, C₃-C₂₀-alkylene or a moiety of formula 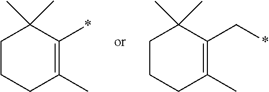
(the * shows where the moiety is attached),
R₁ is H, CH₂OR₂, wherein
R₂ is H or —(CO)R₃, wherein R₃ is a C₁-C₁₈-alkyl, or
R₁ is a moiety of formula 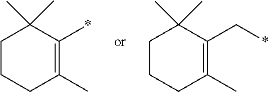
(the * shows where the moiety is attached),
characterized in that a compound of formula (II) 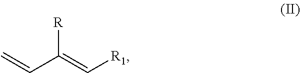
wherein R and R₁ have the same meanings as defined above
is reacted with SO₂, wherein the process is carried out in at least one solvent, wherein the solvent is at least one alcohol.

2. Process according to claim 1, wherein
R is C₁-C₂-alkyl, C₃-C₁₆-alkylene or 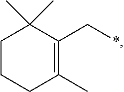
(the * shows where the moiety is attached),
R₁ is H, CH₂OR₂, wherein
R₂ is H or —(CO)R₃, wherein R₃ is a C₁-C₁₆-alkyl, or
R₁ is a moiety of formula 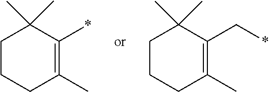
(the * shows where the moiety is attached).

3. Process according to claim 1, wherein compounds of formula (I′) 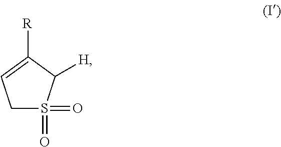
wherein R is CH₃, C₁₀-C₁₆-alkylene or 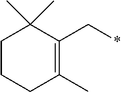 [SMILES: *CC1=C(C)CCCC1(C)C]
(the * shows where the moiety is attached).
are produced.

4. Process according to claim 1, wherein compounds of formula (I″) 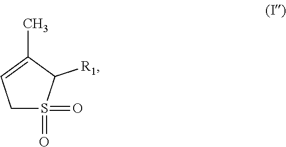
wherein R₁ is CH₂OR₂, wherein
R₂ is H or —(CO)R₃, wherein R₃ is a C₁-C₁₆-alkyl, or
R₁ is a moiety of formula 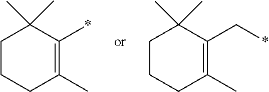
(the * shows where the moiety is attached),
are produced.

5. Process according to claim 1, wherein the following compounds are produced

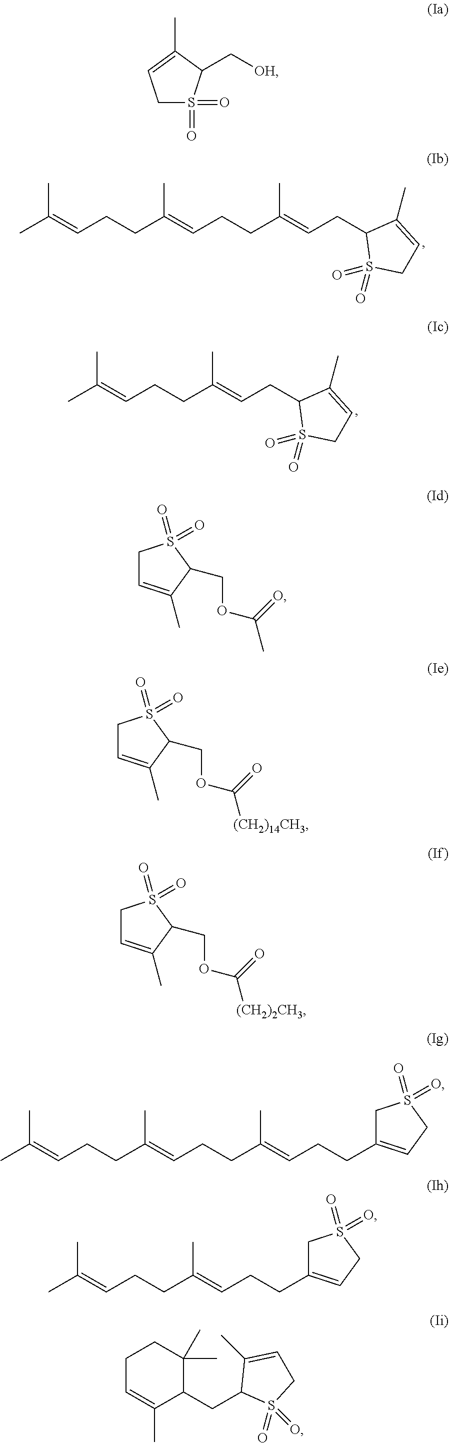

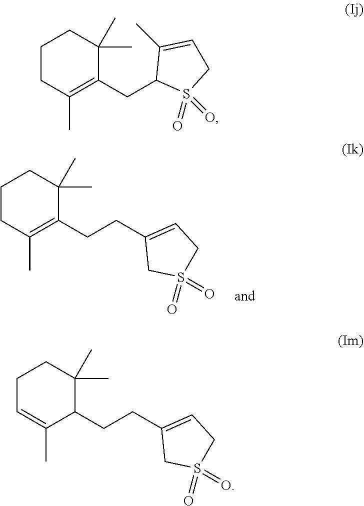

6. Process according to claim 1, wherein the alcohol is chosen from the group consisting of methanol, ethanol, 1-propanol, 2-propanol, tert-butanol and ethylene glycol.

7. Process according to claim 1, wherein the process is carried out at a temperature range of from 20° C. to 125° C.

8. Process according to claim 1, wherein the process is carried out under increased pressure.

9. Process according to claim 8, wherein the gauge pressure is between 0.5 to 5 barg.

10. Process according to claim 1 wherein the process is carried out in the presence of at least one stabilizer.

11. Process according to claim 10, wherein the at least one stabilizer is chosen from the group consisting of butylated hydroxytoluene (BHT), phenothiazine, 4-tert-butylcatechol and tocopherol.

12. Compounds [chemical structure]
